(19) United States
(12) Patent Application Publication
(10) Pub. No.: US 2001/0039369 A1
(43) Pub. Date: Nov. 8, 2001

(54) Blood pump system

(76) Inventor: Alexandre N. Terentiev, Lexington, KY (US)

Correspondence Address: Mark A. Taylor, Stoll, Keenon & Park, LLP, Suite 1000, 201 East Main Street, Lexington, KY, 40507-1380, US

(21) Appl. No.: 09/776,429

(22) Filed: Feb. 2, 2001

Related U.S. Application Data
(63) Continuation-in-part of application No. 09/460,600, filed on Dec. 14, 1999.
(63) Continuation-in-part of application No. 09/724,815, filed on Nov. 28, 2000.
(60) Provisional application No. 60/243,421, filed on Oct. 25, 2000.

(51) Int. Cl. A61M 1/12

(52) U.S. Cl. 600/16

(57) ABSTRACT

The present invention is directed to a blood pumping system that includes two portions, including a pump portion capable of use inside a living body and a drive portion for disposition outside the living body. The drive portion includes a pump drive system, including an assembly configured to cause the impeller to rotate in addition to an assembly configured to levitate the impeller. The present invention includes two separate versions of the pump drive system, including a first embodiment having a high temperature superconductor to levitate the impeller into a predetermined and substantially translationally fixed position spaced from said pump housing, and a rotating, motor driven magnet system to cause the impeller to rotate, and a second embodiment that incorporates a rotating superconductor, which will both levitate and rotate the impeller. Each embodiment of the pump drive system includes a cryostat vessel in which the superconductor is disposed for confining the superconductor in a liquid nitrogen environment to therefore maintain its temperature at 77 K in order to cause the superconductor to levitate magnets within the pump.

### CROSS REFERENCE TO RELATED APPLICATIONS

The present application is a continuation-in-part of application Ser. No. 09/460,600, filed Dec. 14, 1999, and of application Ser. No. 09/724,815, filed Nov. 28, 2000. This application also claims the benefit of the filing date of U.S. Provisional Patent Application Ser. No. 60/243,421, filed Oct. 25, 2000.

### BACKGROUND OF THE INVENTION

The present invention relates broadly to medical fluid pumping systems including blood pumps for internal or external use and, more particularly, to a two-portion blood pump system including an internal pump portion and an external drive portion wherein the pump impeller is levitated and stabilized against translational movement using at least one superconductor mounted in the external drive portion. Human heart problems sometimes give rise to the need for a supplemental or replacement pump to cause blood to flow throughout the body.

One known type of pump used for this purpose is a centrifugal pump, an example of which is available commercially as the BIO-PUMP®, from Medtronics, Inc. of Minneapolis, Minn. Blood pumps may be configured as a two-piece unit, including a pump portion and a drive portion. The separate, external drive portion drives the impeller within the internal pump portion using a magnetic coupling effect. There, rotating magnets within the drive unit magnetically couple with magnets attached to the impeller across a skin boundary wherein rotation of the drive magnets causes similar rotation of the impeller magnets, and consequently, the impeller, thereby causing blood to be pumped and to therefore flow.

The use of conventional pumps with bearings that generate heat due to friction can damage blood cells, and therefore, a pump using magnetic bearings including a levitating impeller is preferred. Electromagnetic or permanent magnet types of couplings can be used to levitate the impeller vertically within its housing, but tend to be unstable with regard to side-to-side, translational movement of the impeller. Therefore, external contact bearings are generally used to stabilize the impeller.

As may be expected, among the problems associated with this type of pump system is the generation of heat within the pump, which can damage cells within the flowing blood.

It would therefore be desirable to incorporate a superconductor for levitation purposes in such a separate pump and drive system. Such a levitated, superconductor-based bearing is described in Terentiev, et al., U.S. Pat. No. 5,567,672, which is herein incorporated by reference. Additionally, Terentiev, U.S. patent application Ser. No. 09/460,600, filed Dec. 14, 1999, a parent application of the present application and which is also incorporated by reference, describes the use of a superconductor for levitating a magnetic bearing. An improved superconducting levitation mixing or pumping system driven by a rotating superconductor is disclosed in Terentiev, U.S. patent application, Ser. No. 09/724,815, filed Nov. 28, 2000, another parent application of the present application, which is likewise incorporated herein by reference.

The present application extends these concepts into a blood pump system which may be used as a substitute or supplement for a natural heart in an internal/external arrangement or which may be used completely external of the body, as necessary.

### SUMMARY OF THE INVENTION

The present invention is directed to a blood pumping system that includes two portions. A first, pump portion includes a pump housing containing a impeller configured for being driven and levitated within its housing. The pump portion may be used internally within skin boundaries or externally as required.

A second, drive portion is included in the present invention and is configured for being strapped to the body of a user when used with an internal pump portion. The drive portion includes a pump drive system, including an assembly configured to cause the impeller to rotate in addition to an assembly configured to levitate the impeller into a predetermined and substantially translationally fixed position spaced from the pump housing.

The present invention includes two separate versions of the pump drive system, which necessitates two separate versions of the pump with respect to its associated drive components.

Basically, a first embodiment of the pump drive system includes a high temperature superconductor to levitate the impeller and a rotating, motor driven magnet system to cause the impeller to rotate. A second embodiment of the pump drive system incorporates a rotating superconductor, which will both levitate and rotate the impeller. The internal configuration of the pump and impeller system is dependent upon the type of drive system chosen.

Each embodiment of the pump drive system includes a cryostat vessel in which the superconductor is disposed for confining the superconductor in a liquid nitrogen environment to therefore maintain its temperature at 77 K in order to cause the superconductor to levitate magnets within the pump. The cryogenic fluid is replaceable by a user of the device in order to maintain the proper operational temperature of the superconductor. This arrangement provides a separate cooling system for the superconductor to isolate the low temperature environment of the superconductor from the pumped fluid.

According to one preferred embodiment of the present invention, a superconductor is a melt-textured yttrium-barium-copper oxide compound. More specifically, the material is melt-textured YBa₂Cu₃O_{X}. This material exhibits high performance in terms of critical current density under a magnetic field at 77K and is thus well suited for magnetic bearings. Nevertheless, it should be understood that any superconducting material with a critical temperature of greater than 77 K could be used.

More specifically, and in furtherance of the above general description, a blood pump system is provided for use in providing blood circulation within a living body for reduced hemolysis effects. According to a first preferred embodiment of the present invention, a blood pump system is provided and includes a pump portion for receiving blood from and delivering blood to a circulatory system associated with the living body. The pump portion includes a housing having a coupling surface formed thereon with the housing defining a pumping chamber and having an inlet formed thereon for receiving influent blood and an outlet formed thereon for delivering effluent blood. The pump portion further includes a movable pumping member, which can be an impeller, disposed in the pumping chamber for movement to cause blood pumping action and at least one magnet operatively associated with the pumping member for movement therewith.

The blood pump system also includes a drive portion thermally isolated from the pump portion for driving the pumping member to cause blood pumping action. It should be understood that the term “drive portion” refers to the levitating assembly and the drive assembly contained in a common housing and mounted adjacent the pump portion. It will be appreciated that only the superconductor or superconductors need be thermally isolated from the pump portion, and that the magnetic drive assembly can be at room temperature. The drive portion is configured for operational disposition adjacent the pump portion and is thermally isolated therefrom. The drive portion includes a drive housing defining a drive chamber therein and having a coupling surface formed thereon for operational disposition adjacent the coupling surface formed on the pump housing, an insulated cryostat vessel disposed in the drive chamber for containment of a cryogen for superconductor cooling, and a superconductor member for cooperation with the at least one magnet for levitating the pumping member. The superconductor member is disposed in the cryostat vessel at a position operationally adjacent the coupling surface of the drive housing in thermal communication with cryogen within the vessel for being cooled thereby to an operating temperature for levitating the pumping member into a predetermined and substantially translationally fixed position spaced from the pump housing. The drive portion further includes a motive device operationally coupled with the at least one magnet in the pump portion for causing movement thereof and thereby causing movement of the pumping member to thereby cause blood pumping action in the pump portion.

Preferably, the pump portion of the present invention may also be configured for implantation within the living body and the drive portion may include an arrangement for retaining the drive portion against the living body in operational engagement with the pump portion. The arrangement for retaining includes a strap member and a fastener arrangement. This can include a belt-like strap with a snap fastener, a hook-and-loop type fastener or other fastening assembly.

According to one preferred drive system, the pump be driven utilizing a coupling effect between the superconductor member and a magnet in the pump portion. The present invention may therefore be further defined wherein the pumping member is formed as an impeller, and the at least one magnet is polarized in an asymmetrical relationship with an axis of rotation of the impeller. The motive device may include an electric motor operationally attached to the superconductor member and powered from a power source, the superconductor member operationally coupling the motive device to the at least one magnet for causing rotational motion thereof to thereby cause rotational motion of the impeller to cause blood pumping action. The power source may be a battery.

The present invention may preferably further include a force transmitting assembly for operationally associating the electric motor with the superconductor member. Alternately, the present inventions may preferably include a direct drive arrangement for operationally associating the electric motor with the superconductor member wherein the superconductor member rotates in a one-to-one relationship with the electric motor. As used herein the term “operationally associating” and variations thereof means that operation on or by one element affects another element “operationally associated” therewith, and indicates that a direct mechanical connection between the elements in question may be prevented by intervening structure without departing from the desired function.

According to another preferred drive system, the present blood pump may also be driven by a magnetic coupling arrangement utilizing one or more permanent magnets in the pump portion in cooperation with one or more permanent magnets in the drive portion. According to the present invention, the at least one magnet includes at least one motion coupler magnet mounted to the pumping member for movement therewith and at least one levitation coupler magnet mounted to the pumping member for levitational interaction with the superconductor member and the drive portion includes at least one drive coupling magnet disposed in the drive housing for magnetically coupling with the at least one motion coupler magnet whereby movement of the at least one drive coupling magnet causes movement of the at least one motion coupler magnet and the pumping member to cause blood pumping action.

Further, the motive device may preferably include an electric motor powered from a power source and the blood pump system may preferably further include a force transmitting assembly for operationally associating the electric motor with the drive coupling magnet. The power source is a battery.

Alternately, the motive device may preferably include an electric motor powered from a power source and the blood pump system further includes a direct drive arrangement for operationally associating the electric motor with the drive coupling magnet wherein the drive coupling magnet rotates in a one-to-one relationship with the electric motor. The power source may be a battery.

Regarding specific material use, the cryogen is preferably liquid nitrogen. The superconductor member may preferably be formed from a melt-textured yttrium-barium-copper oxide compound.

Another version of the blood pump system according to another preferred embodiment thereof is characterized by a magnet drive coupling. According to the present invention, a blood pump system for use in providing blood circulation within a living body for reduced hemolysis effects is provided. The blood pump system includes a pump portion for receiving blood from and delivering blood to a circulatory system associated with the living body. The pump portion includes a pump housing having a coupling surface formed thereon, with the pump housing defining a pumping chamber and having an inlet formed thereon for receiving influent blood and an outlet formed thereon for delivering effluent blood. The pump portion also includes a movable pumping member disposed in the pumping chamber for movement to cause blood pumping action, at least one motion coupler magnet operatively associated with the pumping member for movement therewith, and at least one levitation coupler magnet operatively associated with the pumping member for levitation thereof.

The drive portion is thermally isolated from the pump portion and is used for driving the pumping member to cause blood pumping action, the drive portion being configured for operational disposition adjacent the pump portion. The drive portion includes a drive housing having a coupling surface formed thereon for operational disposition adjacent the coupling surface formed on the pump housing, the drive housing defining a drive chamber therein and an insulated cryostat vessel disposed in the drive chamber for containment of a cryogen for superconductor cooling. A superconductor member is included for cooperation with the at least one levitation coupler magnet for levitating the pumping member into a predetermined and substantially translationally fixed position spaced from the pump housing. The superconductor member is disposed in the cryostat vessel at a position operationally adjacent the coupling surface of the drive housing in thermal communication with cryogen within the vessel for being cooled thereby to an operating temperature for levitating the pumping member. At least one drive coupling magnet is disposed in the drive housing for magnetically coupling with the at least one motion coupler magnet whereby movement of the at least one drive coupling magnet causes movement of the at least one motion coupler magnet and the pumping member. A motive device is operationally connected to the drive coupling magnet for causing movement thereof and thereby causing movement of the pumping member to thereby cause blood pumping action in the pump portion.

Preferably, the pump portion is configured for implantation within the living body and the drive portion includes an arrangement for retaining the drive portion against the living body in operational engagement with the pump portion. It is preferred that the arrangement for retaining includes a strap member and a fastener arrangement.

Further, the motive device may preferably include an electric motor operationally attached to the at least one drive coupling magnet and powered by a power source for causing rotational motion thereof to thereby cause rotational motion of the at least one motion coupler magnet and the pumping member to cause blood pumping action. The power source may be a battery.

Another preferred embodiment of the present invention is characterized by a superconductor-based drive coupling. According to this embodiment, a blood pump system for use in providing blood circulation within a living body for reduced hemolysis effects is provided. The blood pump system includes a pump portion for receiving blood from and delivering blood to a circulatory system associated with the living body. The pump portion includes a pump housing having a coupling surface formed thereon, the pump housing defining a pumping chamber and having an inlet formed thereon for receiving influent blood and an outlet formed thereon for delivering effluent blood. A movable pumping member is disposed in the pumping chamber for movement to cause blood pumping action. At least one coupler magnet is operationally associated with the pumping member for movement therewith and being polarized in an asymmetrical relationship with the superconductor member.

A drive portion is thermally isolated from the pump portion and is provided for driving the pumping member to cause blood pumping action, the drive portion being configured for operational disposition adjacent the pump portion and being thermally isolated therefrom. The drive portion includes a drive housing having a coupling surface formed thereon for operational disposition adjacent the coupling surface formed on the pump housing. The drive housing defines a drive chamber therein. An insulated cryostat vessel is disposed in the drive chamber for containment of a cryogen for superconductor cooling. A superconductor member is provided for cooperation with the at least one coupler magnet for levitating the pumping member into a predetermined and substantially translationally fixed position spaced from the pump housing and driving the pumping member into blood pumping action. The superconductor member is disposed in the cryostat vessel at a position operationally adjacent the coupling surface of the drive housing in thermal communication with cryogen within the vessel for being cooled thereby to an operating temperature for coupling with the at least one coupler magnet for levitating the pumping member into the predetermined and substantially translationally fixed position spaced from the pump housing. A motive device is operationally connected to the superconductor member for causing movement thereof and thereby causing movement of the pumping member to thereby cause blood pumping action in the pump portion.

It is preferred that the motive device includes an electric motor operationally attached to the at least one superconductor member for causing movement thereof to thereby cause movement of the at least one coupler magnet and the pumping member to cause blood pumping action.

It is further preferred that the pump portion is configured for implantation within the living body and the drive portion includes an arrangement for retaining the drive portion against the living body in operational engagement with the pump portion. Preferably, the arrangement for retaining includes a strap member and a fastener arrangement.

More specifically, the pumping member is preferably formed as an impeller, the at least one coupler magnet is polarized in an asymmetrical relationship with an axis of rotation of the impeller, and the motive device includes an electric motor operationally attached to the superconductor member and powered from a power source, the superconductor member operationally coupling the motive device to the at least one magnet for causing rotational motion thereof to thereby cause rotational motion of the at least one magnet and the pumping member to cause blood pumping action. The power source is preferably a battery.

The blood pump system may also include a force transmitting assembly for operationally associating the electric motor with the superconductor member. Alternately, the blood pump system may include a direct drive arrangement for operationally associating the electric motor to the superconductor member wherein the superconductor member rotates in a one-to-one relationship with the electric motor.

The present invention may also be described in terms of its two main, separate individual components, including a blood pump assembly and a separate pump driving assembly. To that end, a blood pump assembly for use in providing blood circulation within a living body for reduced hemolysis effects is provided. The blood pump assembly is configured for being driven by a superconductor-based pump driving assembly thermally isolated from the blood pump assembly. The blood pump assembly includes a pump housing having a coupling surface formed thereon, the pump housing defining a pumping chamber and having an inlet formed thereon for receiving influent blood and an outlet formed thereon for delivering effluent blood. A movable pumping member is disposed in the pumping chamber for levitation by a superconductor disposed in the pump driving assembly for movement to cause blood pumping action. At least one magnet is operatively associated with the pumping member for movement therewith. The at least one magnet is magnetically coupleable to a drive element in the pump driving assembly and drivable into movement thereby.

Preferably, the blood pump assembly is configured for implantation within the living body with the pump driving assembly including means for retaining the pump driving assembly against the living body in operational engagement with the blood pump assembly.

The pump assembly can be driven by either a superconductor-based drive assembly or a permanent magnet-based drive assembly. For a superconductor-based drive assembly, it is preferred that the pumping member is formed as an impeller, the at least one magnet is polarized in an asymmetrical relationship with an axis of rotation of the impeller for cooperation with a superconductor member in the pump driving assembly for operationally coupling a motive device in the pump driving assembly to the at least one magnet for causing rotational motion thereof to thereby cause rotational motion of the pumping member to cause blood pumping action.

For a permanent magnet-based drive assembly, it is preferred that the pump assembly includes at least one motion coupler magnet mounted to the pumping member for movement therewith and at least one levitation coupler magnet operationally associated with the pumping member for cooperation with at least one drive coupling magnet disposed in the pump driving assembly for magnetically coupling with the at least one motion coupler magnet whereby movement of the at least one drive coupling magnet causes movement of the at least one motion coupler magnet and the pumping member to cause blood pumping action.

The pump driving assembly can be one of two basic types, as mentioned above. Accordingly, a blood pump driving assembly is provided for use in conjunction with a pump assembly for providing blood circulation within a living body for reduced hemolysis effects. The blood pump driving assembly is thermally isolated from the pump assembly for driving a pumping member therein to cause blood pumping action, the blood pump driving assembly being configured for operational disposition adjacent the pump assembly while remaining thermally isolated therefrom. The blood pump driving assembly includes a drive housing defining a drive chamber therein and having a coupling surface formed thereon for operational disposition adjacent a coupling surface formed on the pump assembly. Further, an insulated cryostat vessel is disposed in the drive chamber for containment of a cryogen for superconductor cooling. A superconductor member is provided for cooperation with at least one magnet disposed in the pump assembly for levitating the pumping member into a predetermined and substantially translationally fixed position spaced from the pump housing. The superconductor member is disposed in the cryostat vessel at a position operationally adjacent the coupling surface of the drive housing in thermal communication with cryogen within the vessel for being cooled thereby to an operating temperature for levitating the pumping member. A motive device is provided and is operationally coupleable with the at least one magnet in the pump assembly for causing movement thereof and thereby causing movement of the pumping member to thereby cause blood pumping action in the pump assembly.

It is preferred that the associated pump assembly is configured for implantation within the living body and the blood pump driving assembly includes an arrangement for retaining the blood pump driving assembly against the living body in operational engagement with the pump assembly. Preferably, the arrangement for retaining includes a strap member and a fastener arrangement.

According to the superconductor-based blood pump drive assembly, it is preferred that the motive device includes an electric motor powered from a power source and operationally associated with the superconductor member, the superconductor member being operationally coupleable to the at least one magnet for causing motion thereof to thereby cause motion of the pumping member to cause blood pumping action. Preferably, the power source is a battery.

It is further preferred that this embodiment of the present invention include a force transmitting assembly for operationally associating the electric motor with the superconductor member. Alternately, it is similarly preferred that the present invention include a direct drive arrangement for operationally associating the electric motor with the superconductor member wherein the superconductor rotates in a one-to-one relationship with the electric motor.

According to a permanent magnet-based blood pump drive assembly, the blood pump driving assembly includes at least one drive coupling magnet disposed in the drive housing for magnetically coupling with at least one motion coupler magnet disposed in the pump assembly and operationally associated with the pumping member whereby movement of the at least one drive coupling magnet causes movement of the at least one motion coupler magnet and the pumping member to cause blood pumping action.

It is further preferred that the motive device includes an electric motor powered from a power source and the blood pump driving assembly further includes a force transmitting assembly for operationally attaching the electric motor to the drive coupling magnet. Preferably, the power source is a battery.

Alternately, it is preferred that the motive device includes an electric motor powered from a power source and the blood pump driving assembly further includes a direct drive arrangement for operationally attaching the electric motor to the drive coupling magnet wherein the drive coupling magnet rotates in a one-to-one relationship with the electric motor. Preferably, the power source is a battery.

It is preferred that the cryogen is liquid nitrogen, and that the superconductor member is formed from a melt-textured yttrium-barium-copper oxide compound.

More specifically, a permanent magnet-based blood pump drive assembly is provided for use in conjunction with a pump assembly for providing blood circulation within a living body for reduced hemolysis effects. The blood pump driving assembly is thermally isolated from the pump assembly for driving a pumping member therein to cause blood pumping action. The blood pump driving assembly is configured for operational disposition adjacent the pump assembly while remaining thermally isolated therefrom. The blood pump drive assembly includes a drive housing having a coupling surface formed thereon for operational disposition adjacent the coupling surface formed on the pump housing, the drive housing defining a drive chamber therein. An insulated cryostat vessel is disposed in the drive chamber for containment of a cryogen for superconductor cooling. A superconductor member is provided for cooperation with at least one levitation coupler magnet for levitating the pumping member into a predetermined and substantially translationally fixed position spaced from the pump housing, the superconductor member is being disposed in the cryostat vessel at a position operationally adjacent the coupling surface of the drive housing in thermal communication with cryogen within the vessel for being cooled thereby to an operating temperature for levitating the pumping member. At least one drive coupling magnet is disposed in the drive housing for magnetically coupling with a motion coupler magnet disposed in the pump assembly whereby movement of the at least one drive coupling magnet causes movement of the at least one motion coupler magnet and the pumping member. A motive device is operationally associated with the drive coupling magnet for causing movement of the motion coupler magnet and thereby causing movement of the pumping member to thereby cause blood pumping action in the pump assembly.

It is preferred that the pump assembly is configured for implantation within the living body and the blood pump driving assembly includes an arrangement for retaining the blood pump driving assembly against the living body in operational engagement with the pump assembly. Preferably, the arrangement for retaining includes a strap member and a fastener arrangement.

It is further preferred that the motive device includes an electric motor powered from a power source and the blood pump driving assembly further includes a force transmitting assembly for operationally attaching the electric motor to the drive coupling magnet. Preferably, the power source is a battery.

Preferably, the motive device includes an electric motor powered from a power source and the blood pump driving assembly further includes a direct drive arrangement for operationally associating the electric motor with the drive coupling magnet wherein the drive coupling magnet rotates in a one-to-one relationship with the electric motor. It is preferred that the power source is a battery.

Preferentially, the cryogen is liquid nitrogen, and the superconductor member is formed from a melt-textured yttrium-barium-copper oxide compound.

The blood pump assembly can also include a superconductor-based drive for use in conjunction with a pump assembly for providing blood circulation within a living body for reduced hemolysis effects, the blood pump driving assembly being thermally isolated from the pump assembly for driving a pumping member therein to cause blood pumping action. The blood pump driving assembly is configured for operational disposition adjacent the pump assembly while remaining thermally isolated therefrom. The blood pump driving assembly includes a drive housing having a coupling surface formed thereon for operational disposition adjacent a coupling surface formed on the pump housing, the drive housing defining a drive chamber therein. An insulated cryostat vessel is disposed in the drive chamber for containment of a cryogen for superconductor cooling. A superconductor member is provided for cooperation with at least one coupler magnet operationally associated with the pumping member for levitating the pumping member into a predetermined and substantially translationally fixed position spaced from the pump housing and driving the pumping member into blood pumping action, the superconductor member being disposed in the cryostat vessel at a position operationally adjacent the coupling surface of the drive housing in thermal communication with cryogen within the vessel for being cooled thereby to an operating temperature for levitating the pumping member. A motive device is operationally connected to the superconductor member for causing movement thereof and thereby causing movement of the pumping member to thereby cause blood pumping action in the pump portion.

Preferably, the pump assembly is configured for implantation within the living body and the blood pump driving assembly includes an arrangement for retaining the blood pump driving assembly against the living body in operational engagement with the pump assembly. It is preferred that the arrangement for retaining includes a strap member and a fastener arrangement.

It is preferred that the motive device includes an electric motor powered from a power source and the blood pump driving assembly further includes a force transmitting assembly for operationally attaching the electric motor to the superconductor member. Preferably, the power source is a battery.

Alternately, it is preferred that the motive device includes an electric motor powered from a power source and the blood pump driving assembly further includes a direct drive arrangement for operationally associating the electric motor with the superconductor member wherein the drive coupling magnet rotates in a one-to-one relationship with the electric motor. Preferably, the power source is a battery.

It is further preferred that the cryogen is liquid nitrogen, and the superconductor member is formed from a melt-textured yttrium-barium-copper oxide compound.

By the above, the present invention provides a reliable, stable pump that can operate on delicate fluid, such as human blood, without damaging the fluid, e.g., from bearing heat.

### BRIEF DESCRIPTION OF THE DRAWINGS

FIG. 1 is a diagrammatic, cutaway view of a blood pump system according to one preferred embodiment of the present invention;

FIG. 2 is a diagrammatic, cutaway view of a second preferred embodiment of the present invention;

FIG. 3 is a diagrammatic plan view of the magnet and superconductor relationship within the blood pump system illustrated FIG. 2;

FIG. 4 is a diagrammatic, cutaway view of a blood pump system according to another preferred embodiment of the present invention; and

FIG. 5 is a diagrammatic, cutaway view of another preferred embodiment of the present invention.

### DESCRIPTION OF THE PREFERRED EMBODIMENTS

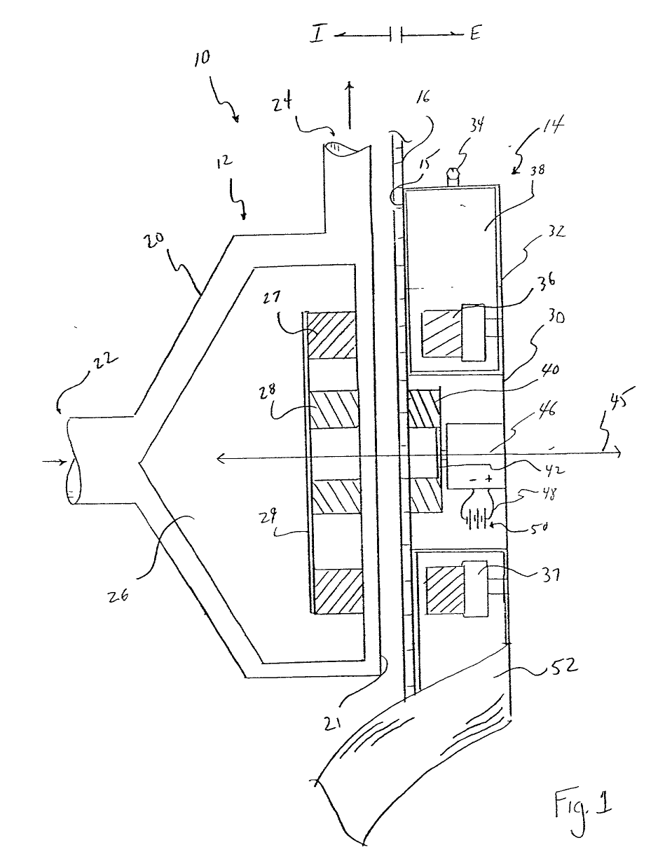

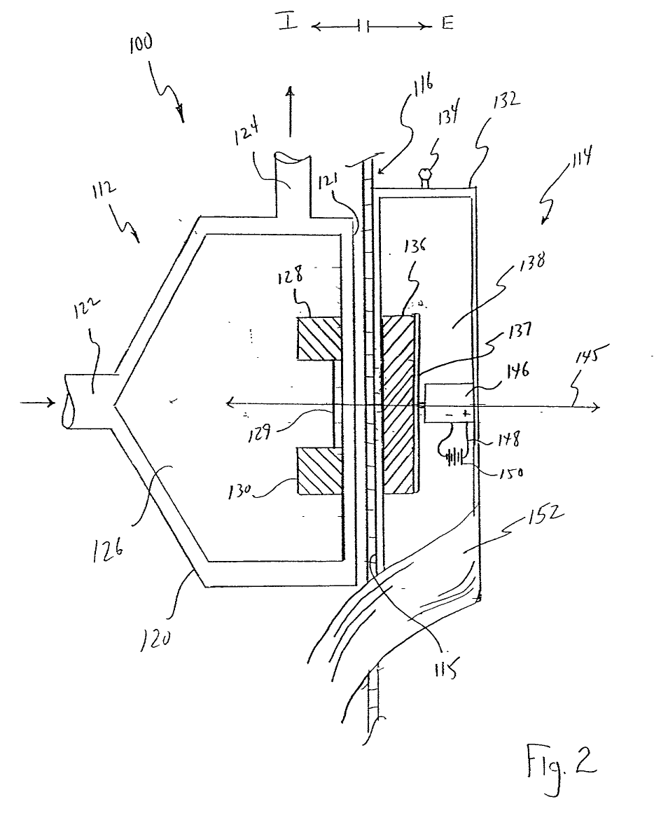

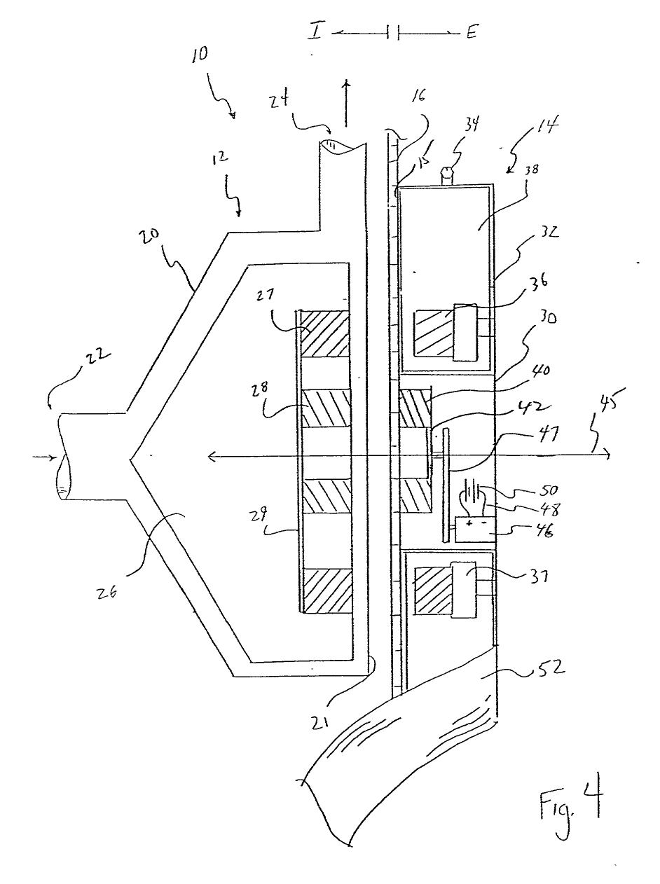

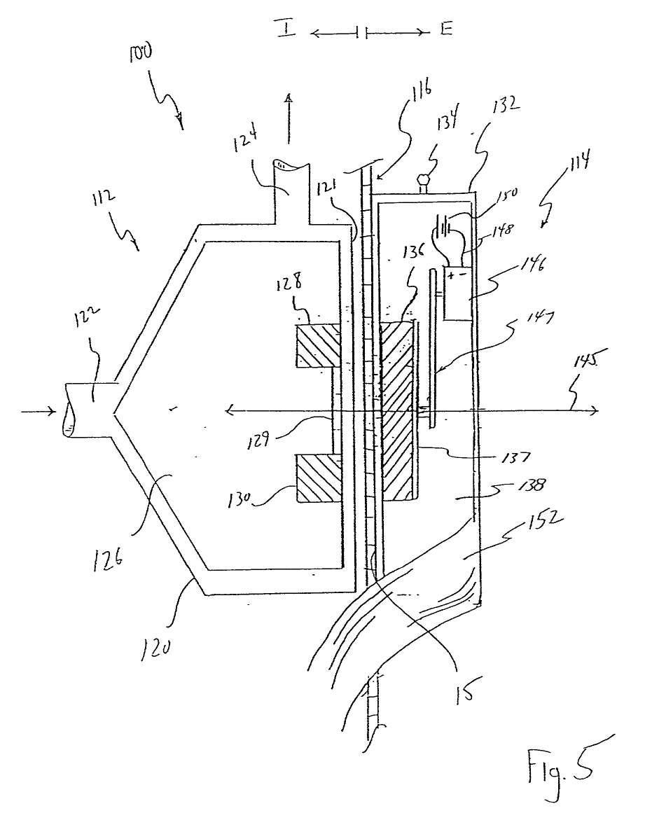

Turning now to the drawings, and more particularly to FIGS. 1 and 2, two separate embodiments of the present inventions are illustrated. Each embodiment will be discussed separately in detail. The first embodiment, illustrated in FIG. 1 provides superconductor levitation of a pump impeller within a heart pump, as explained in greater detail hereinafter. Importantly, the superconductor will levitate the impeller into a predetermined and substantially translationally fixed position spaced from the pump housing. Driven, rotatable magnets in the drive portion couple with magnets attached to the impeller in the pump portion to rotate the impeller. The second embodiment, illustrated in FIG. 2, includes a rotatable superconductor that will both levitate the impeller and cause the impeller to rotate. As will be seen in greater detail hereinafter, FIGS. 4 and 5 illustrate an alternative drive system to the direct drive illustrated in FIGS. 1 and 2.

With reference to FIG. 1, a blood pump system is illustrated generally at 10 and includes a pump portion 12 and a drive portion 14. The pump portion 12 may be disposed internally, within a living body or externally as necessary and as will be apparent to those skilled in using such devices. FIG. 1 illustrates the pump portion 12 disposed internally, within a skin boundary 16. Arrows I and E, illustrate the areas internal and external of the skin boundary 16. The pump portion 12 includes a pump housing 20, which is formed generally as a domed container having a domed impeller 26 disposed therein. A tubular inlet 22 is formed on a peaked portion of the domed housing 20 and a tubular outlet 24 extends from one sidewall of the pump housing 20. Arrows at the inlet 22 and the outlet 24 indicate flow through the pump respectively. A coupling surface 21 is formed on the pump housing 20 to assist in operational alignment of the pump housing 20.

It should be understood by those skilled in the art that the use of a centrifugal pump as a blood pump is a known practice and the diagrammatic view of the pump and drive system according to the present invention is for illustrative purposes only and is not intended to realistically depict an actual pump. It should also be appreciated by those skilled in the pumping art that the diagrammatic views of the present system are sufficient to allow one of ordinary skill in this art to practice the invention without undue experimentation.

Two magnets, or groups of magnets, 27, 28 are provided within the pump housing 20 and are fixed to the impeller 26. The magnets 27, 28 may be formed as a solid, ring-like magnet or may be individual magnets formed in a ring. The magnets will be discussed herein in the singular, as if a single, magnetic ring were used for each function, but it should be understood that the plural may be appropriate for some designs using multiple magnets. Nevertheless, a levitation magnet 27 is disposed outwardly from a drive magnet 28 and these magnets 27, 28 are supported by a structural support member 29. The magnets are supported in general alignment along the impeller 26 in an arrangement adjacent a coupling surface 21 formed on the pump housing 20. It should be noted that virtually any type of permanent magnet may be used including magnets formed from neodymium-iron-boron (NdFeB); Samarium Cobalt (SmCo); the composition of aluminum, nickel and cobalt (Alnico); and ceramic magnets. The levitation magnets 27 have a polarization that is axially symmetrical with respect to the axis of rotation, illustrated at 45. Therefore, during rotation, the levitation magnet 27 does not generate an AC field, allowing frictionless rotation with respect to interaction with the superconductor. The drive magnet 28 is polarized in a nonsymmetrical manner with respect to the axis of rotation 45, thereby enabling magnetic coupling with the drive magnet 40, allowing torque generation upon rotation of the drive magnet 40.

The pump portion 12 is illustrated as being spaced a distance from the skin boundary 16. The spacing reflects internal tissue separating the pump portion 20 from the skin boundary 16 and it should be noted that the pump portion 12 should be disposed adjacent the skin boundary 16, although actual contact may not occur.

The drive portion 14 consists of a drive housing 30 that includes a cryostat vessel 32, as will be explored in greater detail hereinafter. A strap 52 may be used to retain the drive portion 14 against the body of the user in a position to maintain proper drive coupling with the pump portion 12. The strap 52 is illustrative of any device or method to retain the drive portion 14 in operational engagement with the pump portion 12, and may be any combination of belt, strap or strapping material or even adhesive. A coupling surface 15 is formed on the drive housing 30 to assist in proper operational alignment with the coupling surface 21 formed on the pump portion 12.

The insulated cryostat vessel 32 is configured for containment of liquid nitrogen 38 and may be replenished through a fill valve 34. A superconductor ring 36 supported by a support member 37 is mounted within the cryostat vessel 32. The liquid nitrogen 38 maintains the YBCO high temperature superconductor 36 at a critical temperature (T_{c}) of approximately 91 K. Once the superconductor is cooled to less than T_{c} in proximity to the levitation magnet 27, the coupling effect of the superconductor 36 causes the levitation magnet 27 to maintain a fixed distance from the superconductor 36.

In order to rotate the impeller 26, a permanent magnet ring, forming a drive magnet 40 is disposed in the drive housing 30. As with the other magnets, it will be understood that the drive magnet 40 may be a single, permanent magnet ring or may be smaller magnets disposed sufficiently to provide magnetic coupling with the drive magnets 28 within the pump portion 12. A structural support 42 is provided in attachment with the drive magnet 40 and is operatively attached to an electric motor 46 that is powered by a battery 50 through conventional wiring 38. The structural support 42 establishes an operational association between the drive magnet 40 and the electric motor 46. The motor 46 and battery 50 may be housed in the drive housing 30. It will be understood by those skilled in the art that the motor 46 may employ a direct drive system, i.e. directly coupled to the drive magnet 40, such that one rotation of the motor's armature (not shown) will equal one rotation of the magnet 40. Other motor drive arrangements are available as necessary or expedient. FIG. 4 illustrates a belt drive system. There, an endless belt 47 extends around a pinion (not shown) attached to an electric motor 46 and a drive pulley (not shown) attached to the structural support 42. The belt 47 in combination with the pinion and drive pulley form a force transmitting assembly to transmit the motive force of the electric motor 46 to the drive magnet 40. Another force transmitting assembly can include a gear system. Such force transmitting assemblies are known generally for use with electric motor drives.

It will be appreciated that the electric motor 46 and drive magnet 40 should be insulated from the effects of the cryogen 38. FIG. 1 illustrates the motor 46 and drive magnets 40 in a room temperature environment, outside the cryostat vessel 32. In other possible configurations, it should be within the skill of those skilled in this art to isolate the motor 46 and drive magnet 40 from the cryogen 38.

A second embodiment of the present invention is illustrated in FIG. 2. There, a blood pump system with a superconductor drive is illustrated generally at 100 and includes a pump portion 112 and a drive portion 114. As with the first embodiment, the pump portion 112 includes a domed housing 120, with a similarly domed impeller 126 disposed therein. A tubular inlet 122 is formed at the peak of the domed housing 120. A tubular outlet 124 is formed along one housing wall. Arrows at the inlet 122, and outlet 124 illustrate flow through the pump, respectively.

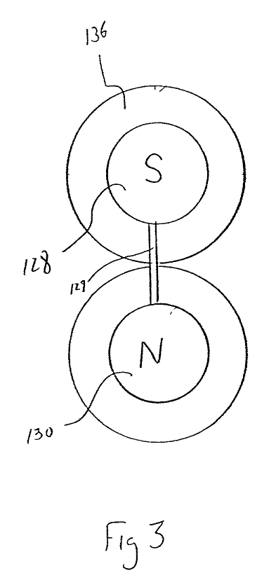

According to the second preferred embodiment, permanent magnets 128, and 130 are disposed with a support 129 extending therebetween. The polarity of these magnets 128, 130 is such that a “North” magnet 128 is disposed adjacent and across the support from an oppositely polarized “South” magnet 130. This arrangement is illustrated generally in FIG. 3. The magnets 128, 130 are mounted within the pump housing 120 adjacent a coupling surface 121.

The drive portion 114 is disposed across a skin boundary 116 from the pump portion 112. As with the other preferred embodiment, it will be apparent to those skilled in the art that the pump portion 112 may be used internally or externally as required. FIG. 2 illustrates by arrows I and E, an internal area containing the pump portion 112 and an external area including the drive portion 114.

The drive portion includes a cryostat vessel 132 which is insulated sufficiently to maintain liquid nitrogen 138 contained therein at a temperature of approximately 77 K in order to cool the superconductor 136 sufficiently to allow levitation to a predetermined and substantially translationally fixed position spaced from said pump housing. The liquid nitrogen 138 may be replenished using a fill valve 134 on the cryostat vessel 132. In should be noted that while liquid nitrogen is the preferred cryogen, other substances may be used as required.

The superconductor 136 can be one solid piece or an array of smaller pieces, formed from the YBCO substance discussed previously and is disposed within the cryostat vessel and supported by a support ring 137. The support ring 137 is operatively attached to an electric motor 146 that is supplied with power from a convential battery 150 through conventional wiring 148. The support ring 137 establishes an operational association between the electric motor 146 and the superconductor 136. As with priorly addressed embodiments, it will be appreciated that the electric motor 140 should be insulated from the effects of the cryogen.

As discussed with reference to another preferred embodiment above, the motor 146 may employ a direct drive system, i.e. directly coupled to the superconductor 136, such that one rotation of the motor armature (not shown) will equal one rotation of the superconductor 136. Other motor drive arrangements are available as necessary or expedient. FIG. 5 illustrates a belt drive system. There, an endless belt 147 extends around a pinion (not shown) attached to an electric motor 146 and a drive pulley (not shown) attached to the support ring 137. The belt 147 in combination with the pinion and drive pulley form a force transmitting assembly to transmit the motive force of the electric motor 146 to the superconductor 136. Another force transmitting assembly can include a gear system. Such force transmitting assemblies are known generally for use with electric motor drives. As with all embodiments of the present invention, the electric motor 140 should be insulated from the effects of the cryogen.

The superconductor 136 is disposed at a position directly opposing the permanent magnets 128, 130 in the pump housing 120 so that rotation of the superconductor 136 causes rotation of the magnet structure 128, 129, 130 within the pump housing 120 and, consequently, rotation of the impeller 126, to cause pumping action. Rotation occurs about an axis of rotation illustrated at 145.

With reference to FIG. 3, the polarity of the magnets 128, 130 contained within the pump portion 112 is illustrated in relationship to the superconductor 136 illustrated in an exaggerated manner in FIG. 3 for clarity. It should be noted that the magnetic field generated by the magnets, 128, 130 attached to the impeller 126 must be axially nonsymmetrical. Therefore, when the impeller 126 rotates relative to the superconductor 136, the superconductor 136 sees a changing field. In the case of an axially nonsymmetrical field, torque will be generated when the superconductor rotates, thereby causing the magnets 128, 130 and the impeller 126 to rotate. According to this embodiment, the magnets 128,130 rotate at the same speed as the superconductor.

The second preferred embodiment of the present invention has the advantage that fewer magnets are required for use in the pump portion 112 and, therefore, the pump portion 112 is generally lighter in the second embodiment as compared to first embodiment.

In operation, and returning to FIG. 1, the pump portion 12 is surgically installed in a body and configured for moving blood therethrough. Operation will be described with respect to the embodiment illustrated in FIG. 1, but it should be understood that operational aspects of the remaining embodiments are similar to those of the FIG. 1 embodiment. The drive portion 14 is strapped to the body of a user using the strap 52. Another use may have the pump portion 12 outside the body during surgery with the drive portion 14 positioned adjacent the pump portion 14 to cause blood pumping action.

The cryogen 38 reduces the temperature of the superconductor to a temperature less than T_{c}, which causes the levitation magnets 27, and, consequently, the impeller 26 to levitate into a predetermined and substantially translationally fixed position spaced from the pump housing 20. The motor 46 is excited into operation which causes rotation of the drive magnets 40 which in turn, through magnetic coupling, causes rotation of the magnet 28 within the pump housing 20 and thereby causes rotation of the impeller 26 to commence blood flow.

Similar operation is associated with the second preferred embodiment illustrated in FIG. 2. There, the cryostat vessel 132 is filled with liquid nitrogen 138, which cools the superconductor 136 to a temperature less than T_{c}. Due to the physical attachment of these magnets 128, 130 to the impeller 126, the impeller 126 levitates into a predetermined and substantially translationally fixed position spaced from the pump housing 120. Excitation of the motor 146 causes rotation of the superconductor, which, in turn, causes rotation of the magnets 128, 130 due to the opposite polarity of the magnets 128, 130. The impeller 126 therefore rotates causing blood to flow.

The present invention provides two embodiments of a blood pump system capable of levitational and rotational stability and reliability heretofore unknown in the medical pumping art. The versatility of the present invention allows the pump to function internally within the living body and externally of the living body as would be useful during surgery.

It will therefore be readily understood by those persons skilled in the art that the present invention is susceptible of a broad utility and application. Many embodiments and adaptations of the present invention other than those herein described, as well as many variations, modifications and equivalent arrangements, will be apparent from or reasonably suggested by the present invention and the foregoing description thereof, without departing from the substance or scope of the present invention. Accordingly, while the present invention has been described herein in detail in relation to its preferred embodiment, it is to be understood that this disclosure is only illustrative and exemplary of the present invention and is made merely for purposes of providing a full and enabling disclosure of the invention. The foregoing disclosure is not intended or to be construed to limit the present invention or otherwise to exclude any such other embodiments, adaptations, variations, modifications and equivalent arrangements, the present invention being limited only by the claims appended hereto and the equivalents thereof.

I claim:

1. A blood pump system for use in providing blood circulation within a living body for reduced hemolysis effects, said blood pump system comprising:
a pump portion for receiving blood from and delivering blood to a circulatory system associated with the living body, said pump portion including:
a pump housing having a coupling surface formed thereon, said pump housing defining a pumping chamber and having an inlet formed thereon for receiving influent blood and an outlet formed thereon for delivering effluent blood;
a movable pumping member disposed in said pumping chamber for movement to cause blood pumping action;
at least one magnet operatively associated with said pumping member for movement therewith;
a drive portion thermally isolated from said pump portion for driving said pumping member to cause blood pumping action, said drive portion being configured for operational disposition adjacent said pump portion and being thermally isolated therefrom, said drive portion including:
a drive housing defining a drive chamber therein and having a coupling surface formed thereon for operational disposition adjacent said coupling surface formed on said pump housing;
an insulated cryostat vessel disposed in said drive chamber for containment of a cryogen for superconductor cooling;
a superconductor member for cooperation with said at least one magnet for levitating said pumping member into a predetermined and substantially translationally fixed position spaced from said pump housing, said superconductor member being disposed in said cryostat vessel at a position operationally adjacent said coupling surface of said drive housing in thermal communication with cryogen within said vessel for being cooled thereby to an operating temperature for levitating said pumping member; and
a motive device operationally coupled with said at least one magnet in said pump portion for causing movement thereof and thereby causing movement of said pumping member to thereby cause blood pumping action in said pump portion.

2. A blood pump system according to claim 1 wherein said pump portion is configured for implantation within the living body and said drive portion includes means for retaining said drive portion against the living body in operational engagement with said pump portion.

3. A blood pump system according to claim 2 wherein said means for retaining includes a strap member and a fastener arrangement.

4. A blood pump system according to claim 1 wherein said pumping member is formed as an impeller, said at least one magnet is polarized in an asymmetrical relationship with an axis of rotation of said impeller, and said motive device includes an electric motor operationally associated with said superconductor member and powered from a power source, with said superconductor member operationally coupling said motive device to said at least one magnet for causing rotational motion thereof to thereby cause rotational motion of said impeller to cause blood pumping action.

5. A blood pump system according to claim 4 wherein said power source is a battery.

6. A blood pump system according to claim 4 and including a force transmitting assembly for operationally associating said electric motor with said superconductor member.

7. A blood pump system according to claim 4 and including a direct drive arrangement for operationally associating said electric motor with said superconductor member wherein said superconductor member rotates in a one-to-one relationship with said electric motor.

8. A blood pump system according to claim 1 wherein said at least one magnet includes at least one motion coupler magnet mounted to said pumping member for movement therewith and at least one levitation coupler magnet mounted to said pumping member for levitational interaction with said superconductor member for levitating said pumping member into a predetermined and substantially translationally fixed position spaced from said pump housing, and said drive portion includes at least one drive coupling magnet disposed in said drive housing for magnetically coupling with said at least one motion coupler magnet whereby movement of said at least one drive coupling magnet causes movement of said at least one motion coupler magnet and said pumping member to cause blood pumping action.

9. A blood pump system according to claim 8 wherein said motive device includes an electric motor powered from a power source and said blood pump system further includes a force transmitting assembly for operationally associating said electric motor with said drive coupling magnet.

10. A blood pump system according to claim 9 wherein said power source is a battery.

11. A blood pump system according to claim 8 wherein said motive device includes an electric motor powered from a power source and said blood pump system further includes a direct drive arrangement for operationally associating said electric motor with said drive coupling magnet wherein said drive coupling magnet rotates in a one-to-one relationship with said electric motor.

12. A blood pump system according to claim 11 wherein said power source is a battery.

13. A blood pump system according to claim 1 wherein said cryogen is liquid nitrogen.

14. A blood pump system according to claim 1 wherein said superconductor member is formed from a melt-textured yttrium-barium-copper oxide compound.

15. A blood pump system for use in providing blood circulation within a living body for reduced hemolysis effects, said blood pump system comprising:
a pump portion for receiving blood from and delivering blood to a circulatory system associated with the living body, said pump portion including:
a pump housing having a coupling surface formed thereon, said pump housing defining a pumping chamber and having an inlet formed thereon for receiving influent blood and an outlet formed thereon for delivering effluent blood;
a movable pumping member disposed in said pumping chamber for movement to cause blood pumping action;
at least one motion coupler magnet operatively associated with said pumping member for movement therewith;
at least one levitation coupler magnet operatively associated with said pumping member for levitation thereof;
a drive portion thermally isolated from said pump portion for driving said pumping member to cause blood pumping action, said drive portion being configured for operational disposition adjacent said pump portion and being thermally isolated therefrom, said drive portion including:
a drive housing having a coupling surface formed thereon for operational disposition adjacent said coupling surface formed on said pump housing, said drive housing defining a drive chamber therein;
an insulated cryostat vessel disposed in said drive chamber for containment of a cryogen for superconductor cooling;
a superconductor member for cooperation with said at least one levitation coupler magnet for levitating said pumping member into a predetermined and substantially translationally fixed position spaced from said pump housing, said superconductor member being disposed in said cryostat vessel at a position operationally adjacent said coupling surface of said drive housing in thermal communication with cryogen within said vessel for being cooled thereby to an operating temperature for levitating said pumping member;
at least one drive coupling magnet disposed in said drive housing for magnetically coupling with said at least one motion coupler magnet whereby movement of said at least one drive coupling magnet causes movement of said at least one motion coupler magnet and said pumping member; and
a motive device operationally connected to said drive coupling magnet for causing movement thereof and thereby causing movement of said pumping member to thereby cause blood pumping action in said pump portion.

16. A blood pump system according to claim 15 wherein said pump portion is configured for implantation within the living body and said drive portion includes means for retaining said drive portion against the living body in operational engagement with said pump portion.

17. A blood pump system according to claim 16 wherein said means for retaining includes a strap member and a fastener arrangement.

18. A blood pump system according to claim 15 wherein said motive device includes an electric motor operationally associated with said at least one drive coupling magnet and powered by a power source for causing rotational motion thereof to thereby cause rotational motion of said at least one motion coupler magnet and said pumping member to cause blood pumping action.

19. A blood pump system according to claim 18 wherein said power source is a battery.

20. A blood pump system according to claim 15 wherein said cryogen is liquid nitrogen.

21. A blood pump system according to claim 15 wherein said superconductor member is formed from a melt-textured yttrium-barium-copper oxide compound.

22. A blood pump system for use in providing blood circulation within a living body for reduced hemolysis effects, said blood pump system comprising:
a pump portion for receiving blood from and delivering blood to a circulatory system associated with the living body, said pump portion including:
a pump housing having a coupling surface formed thereon, said pump housing defining a pumping chamber and having an inlet formed thereon for receiving influent blood and an outlet formed thereon for delivering effluent blood;
a movable pumping member disposed in said pumping chamber for movement to cause blood pumping action;
at least one coupler magnet operationally associated with said pumping member for movement therewith and being polarized in an asymmetrical relationship with said superconductor member;
a drive portion thermally isolated from said pump portion for driving said pumping member to cause blood pumping action, said drive portion being configured for operational disposition adjacent said pump portion and being thermally isolated therefrom, said drive portion including:
a drive housing having a coupling surface formed thereon for operational disposition adjacent said coupling surface formed on said pump housing, said drive housing defining a drive chamber therein;
an insulated cryostat vessel disposed in said drive chamber for containment of a cryogen for superconductor cooling;
a superconductor member for cooperation with said at least one coupler magnet for levitating said pumping member into a predetermined and substantially translationally fixed position spaced from said pump housing, and driving said pumping member into blood pumping action, said superconductor member being disposed in said cryostat vessel at a position operationally adjacent said coupling surface of said drive housing in thermal communication with cryogen within said vessel for being cooled thereby to an operating temperature for coupling with said at least one coupler magnet for levitating said pumping member; and
a motive device operationally connected to said superconductor member for causing movement thereof and thereby causing movement of said pumping member to thereby cause blood pumping action in said pump portion.

23. A blood pump system according to claim 22 wherein said motive device includes an electric motor operationally associated with said superconductor member for causing movement thereof to thereby cause movement of said at least one coupler magnet and said pumping member to cause blood pumping action.

24. A blood pump system according to claim 22 wherein said pump portion is configured for implantation within the living body and said drive portion includes means for retaining said drive portion against the living body in operational engagement with said pump portion.

25. A blood pump system according to claim 24 wherein said means for retaining includes a strap member and a fastener arrangement.

26. A blood pump system according to claim 22 wherein said pumping member is formed as an impeller, said at least one coupler magnet is polarized in an asymmetrical relationship with an axis of rotation of said impeller, and said motive device includes an electric motor operationally associated with said superconductor member and powered from a power source, said superconductor member operationally coupling said motive device to said at least one coupler magnet for causing rotational motion thereof to thereby cause rotational motion of said at least one coupler magnet and said pumping member to cause blood pumping action.

27. A blood pump system according to claim 26 wherein said power source is a battery.

28. A blood pump system according to claim 26 and including a force transmitting assembly for operationally associating said electric motor with said superconductor member.

29. A blood pump system according to claim 26 and including a direct drive arrangement for operationally associating said electric motor with said superconductor member wherein said superconductor member rotates in a one-to-one relationship with said electric motor.

30. A blood pump assembly for use in providing blood circulation within a living body for reduced hemolysis effects, said blood pump assembly being configured for being driven by a superconductor-based pump driving assembly thermally isolated from said blood pump assembly and comprising:
a pump housing having a coupling surface formed thereon, said pump housing defining a pumping chamber and having an inlet formed thereon for receiving influent blood and an outlet formed thereon for delivering effluent blood;
a movable pumping member disposed in said pumping chamber for levitation by a superconductor into a predetermined and substantially translationally fixed position spaced from said pump housing, said pumping member being disposed in the pump driving assembly for movement to cause blood pumping action; and
at least one magnet operatively associated with said pumping member for movement therewith, said at least one magnet being magnetically coupleable to a drive element in the pump driving assembly and drivable into movement thereby.

31. A blood pump assembly according to claim 30 wherein said pump assembly is configured for implantation within the living body with the pump driving assembly including means for retaining said pump driving assembly against the living body in operational engagement with said blood pump assembly.

32. A blood pump assembly according to claim 30 wherein said pumping member is formed as an impeller, said at least one magnet is polarized in an asymmetrical relationship with an axis of rotation of said impeller for cooperation with a superconductor member in the pump driving assembly for operationally coupling a motive device in the pump driving assembly to said at least one magnet for causing rotational motion thereof to thereby cause rotational motion of said pumping member to cause blood pumping action.

33. A blood pump assembly according to claim 30 wherein said pump assembly includes at least one motion coupler magnet mounted to said pumping member for movement therewith and at least one levitation coupler magnet operationally associated with said pumping member for cooperation with at least one drive coupling magnet disposed in the pump driving assembly for magnetically coupling with said at least one motion coupler magnet whereby movement of said at least one drive coupling magnet causes movement of said at least one motion coupler magnet and said pumping member to cause blood pumping action.

34. A blood pump driving assembly for use in conjunction with a pump assembly for providing blood circulation within a living body for reduced hemolysis effects, said blood pump driving assembly being thermally isolated from the pump assembly for driving a pumping member therein to cause blood pumping action, said blood pump driving assembly being configured for operational disposition adjacent the pump assembly while remaining thermally isolated therefrom, said blood pump driving assembly comprising:
a drive housing defining a drive chamber therein and having a coupling surface formed thereon for operational disposition adjacent a coupling surface formed on the pump assembly;
an insulated cryostat vessel disposed in said drive chamber for containment of a cryogen for superconductor cooling;
a superconductor member for cooperation with at least one magnet disposed in the pump assembly for levitating the pumping member into a predetermined and substantially translationally fixed position spaced from said pump housing, said superconductor member being disposed in said cryostat vessel at a position operationally adjacent said coupling surface of said drive housing in thermal communication with cryogen within said vessel for being cooled thereby to an operating temperature for levitating the pumping member; and
a motive device operationally coupleable with the at least one magnet in the pump assembly for causing movement thereof and thereby causing movement of the pumping member to thereby cause blood pumping action in the pump assembly.

35. A blood pump driving assembly according to claim 34 wherein the pump assembly is configured for implantation within the living body and said blood pump driving assembly includes means for retaining said blood pump driving assembly against the living body in operational engagement with the pump assembly.

36. A blood pump driving assembly according to claim 35 wherein said means for retaining includes a strap member and a fastener arrangement.

37. A blood pump driving assembly according to claim 34 wherein said motive device includes an electric motor powered from a power source and operationally associated with said superconductor member, said superconductor member being operationally coupleable to the at least one magnet for causing motion thereof to thereby cause motion of the pumping member to cause blood pumping action.

38. A blood pump driving assembly according to claim 37 wherein said power source is a battery.

39. A blood pump driving assembly according to claim 37 and including a force transmitting assembly for operationally associating said electric motor with said superconductor member.

40. A blood pump driving assembly according to claim 37 and including a direct drive arrangement for operationally associating said electric motor with said superconductor member wherein said superconductor rotates in a one-to-one relationship with said electric motor.

41. A blood pump driving assembly according to claim 34 wherein said blood pump driving assembly includes at least one drive coupling magnet disposed in said drive housing for magnetically coupling with at least one motion coupler magnet disposed in the pump assembly and operationally associated with the pumping member whereby movement of said at least one drive coupling magnet causes movement of the at least one motion coupler magnet and the pumping member to cause blood pumping action.

42. A blood pump system according to claim 41 wherein said motive device includes an electric motor powered from a power source and said blood pump driving assembly further includes a force transmitting assembly for operationally associating said electric motor with said drive coupling magnet.

43. A blood pump driving assembly according to claim 34 wherein said power source is a battery.

44. A blood pump driving assembly according to claim 34 wherein said motive device includes an electric motor powered from a power source and said blood pump driving assembly further includes a direct drive arrangement for operationally associating said electric motor with said drive coupling magnet wherein said drive coupling magnet rotates in a one-to-one relationship with said electric motor.

45. A blood pump driving assembly according to claim 44 wherein said power source is a battery.

46. A blood pump driving assembly according to claim 34 wherein said cryogen is liquid nitrogen.

47. A blood pump driving assembly according to claim 34 wherein said superconductor member is formed from a melt-textured yttrium-barium-copper oxide compound.

48. A blood pump driving assembly for use in conjunction with a pump assembly for providing blood circulation within a living body for reduced hemolysis effects, said blood pump driving assembly being thermally isolated from the pump assembly for driving a pumping member therein to cause blood pumping action, said blood pump driving assembly being configured for operational disposition adjacent the pump assembly while remaining thermally isolated therefrom, said drive assembly comprising:
a drive housing having a coupling surface formed thereon for operational disposition adjacent said coupling surface formed on said pump housing, said drive housing defining a drive chamber therein;
an insulated cryostat vessel disposed in said drive chamber for containment of a cryogen for superconductor cooling;
a superconductor member for cooperation with at least one levitation coupler magnet for levitating the pumping member into a predetermined and substantially translationally fixed position spaced from said pump housing, said superconductor member being disposed in said cryostat vessel at a position operationally adjacent said coupling surface of said drive housing in thermal communication with cryogen within said vessel for being cooled thereby to an operating temperature for levitating the pumping member;
at least one drive coupling magnet disposed in said drive housing for magnetically coupling with a motion coupler magnet disposed in the pump assembly whereby movement of said at least one drive coupling magnet causes movement of the at least one motion coupler magnet and the pumping member; and
a motive device operationally connected to the drive coupling magnet for causing movement of the motion coupler magnet and thereby causing movement of the pumping member to thereby cause blood pumping action in the pump assembly.

49. A blood pump driving assembly according to claim 48 wherein the pump assembly is configured for implantation within the living body and said blood pump driving assembly includes means for retaining said blood pump driving assembly against the living body in operational engagement with the pump assembly.

50. A blood pump driving assembly according to claim 49 wherein said means for retaining includes a strap member and a fastener arrangement.

51. A blood pump driving assembly according to claim 49 wherein said motive device includes an electric motor powered from a power source and said blood pump driving assembly further includes a force transmitting assembly for operationally associating said electric motor with said drive coupling magnet.

52. A blood pump driving assembly according to claim 51 wherein said power source is a battery.

53. A blood pump driving assembly according to claim 48 wherein said motive device includes an electric motor powered from a power source and said blood pump driving assembly further includes a direct drive arrangement for operationally associating said electric motor with said drive coupling magnet wherein said drive coupling magnet rotates in a one-to-one relationship with said electric motor.

54. A blood pump driving assembly according to claim 53 wherein said power source is a battery.

55. A blood pump driving assembly according to claim 48 wherein said cryogen is liquid nitrogen.

56. A blood pump driving assembly according to claim 48 wherein said superconductor member is formed from a melt-textured yttrium-barium-copper oxide compound.

57. A blood pump driving assembly for use in conjunction with a pump assembly for providing blood circulation within a living body for reduced hemolysis effects, said blood pump driving assembly being thermally isolated from the pump assembly for driving a pumping member therein to cause blood pumping action, said blood pump driving assembly being configured for operational disposition adjacent the pump assembly while remaining thermally isolated therefrom, said blood pump driving assembly comprising:
a drive housing having a coupling surface formed thereon for operational disposition adjacent a coupling surface formed on the pump housing, said drive housing defining a drive chamber therein;
an insulated cryostat vessel disposed in said drive chamber for containment of a cryogen for superconductor cooling;
a superconductor member for cooperation with at least one coupler magnet operationally associated with the pumping member for levitating the pumping member into a predetermined and substantially translationally fixed position spaced from said pump housing, and for driving the pumping member into blood pumping action, said superconductor member being disposed in said cryostat vessel at a position operationally adjacent said coupling surface of said drive housing in thermal communication with cryogen within said vessel for being cooled thereby to an operating temperature for levitating the pumping member; and
a motive device operationally connected to said superconductor member for causing movement thereof and thereby causing movement of said pumping member to thereby cause blood pumping action in said pump portion.

58. A blood pump driving assembly according to claim 57 wherein the pump assembly is configured for implantation within the living body and said blood pump driving assembly includes means for retaining said blood pump driving assembly against the living body in operational engagement with the pump assembly.

59. A blood pump driving assembly according to claim 58 wherein said means for retaining includes a strap member and a fastener arrangement.

60. A blood pump driving assembly according to claim 57 wherein said motive device includes an electric motor powered from a power source and said blood pump driving assembly further includes a force transmitting assembly for operationally associating said electric motor with said superconductor member.

61. A blood pump driving assembly according to claim 60 wherein said power source is a battery.

62. A blood pump driving assembly according to claim 57 wherein said motive device includes an electric motor powered from a power source and said blood pump driving assembly further includes a direct drive arrangement for operationally associating said electric motor with said superconductor member wherein said drive coupling magnet rotates in a one-to-one relationship with said electric motor.

63. A blood pump driving assembly according to claim 62 wherein said power source is a battery.

64. A blood pump system according to claim 57 wherein said cryogen is liquid nitrogen.

65. A blood pump system according to claim 57 wherein said superconductor member is formed from a melt-textured yttrium-barium-copper oxide compound.
